(19) United States
(12) Patent Application Publication
(10) Pub. No.: US 2024/0164921 A1
(43) Pub. Date: May 23, 2024

(54) IN-VIVO INDWELLING STENT AND STENT DELIVERY SYSTEM

(71) Applicant: TERUMO KABUSHIKI KAISHA, Tokyo (JP)

(72) Inventors: Takashi KITAOKA, Hadano city (JP); Hiroshi YAMAGUCHI, Ebina city (JP); Taishi NIIMI, Hadano city (JP); Yukiko TANABE, Hadano city (JP)

(73) Assignee: TERUMO KABUSHIKI KAISHA, Tokyo (JP)

(21) Appl. No.: 18/426,869

(22) Filed: Jan. 30, 2024

Related U.S. Application Data
(63) Continuation of application No. PCT/JP2022/027025, filed on Jul. 7, 2022.

(30) Foreign Application Priority Data
Aug. 5, 2021 (JP) 2021-128641

(51) Int. Cl. A61F 2/915; A61F 2/95

(52) U.S. Cl. CPC A61F 2/915; A61F 2/95; A61F 2220/0025

(57) ABSTRACT

This in-vivo indwelling stent has annular bodies constituted by a plurality of annular bodies arranged in the axial direction, and connection portions. The plurality of annular bodies includes first pattern annular bodies and second pattern annular bodies that are alternately arranged. The first pattern annular bodies each have an endless zigzag linear shape including one-end-side apexes, other-end-side apexes, and curved linear bodies that connects the apexes and protrudes in a first circumferential direction. The second pattern annular bodies each have an endless zigzag linear shape including one-end-side apexes, other-end-side apexes, and a plurality of curved linear bodies that connects the apexes and protrudes in a second circumferential direction. The one-end-side apex and the other-end-side apex of adjacent annular bodies are connected to each other with the connection portion.

### CROSS-REFERENCES TO RELATED APPLICATIONS

This application is a continuation of International Patent Application No. PCT/JP2022/027025 filed on Jul. 7, 2022, which claims priority to Japanese Patent Application No. 2021-128641 filed on Aug. 5, 2021, the entire content of both of which is incorporated herein by reference.

### TECHNOLOGICAL FIELD

The present invention generally relates to an in-vivo indwelling stent and a stent delivery system used for improving a stenotic site or an occluded site generated in a body lumen such as a blood vessel, a bile duct, a trachea, an esophagus, or a urethra.

### BACKGROUND DISCUSSION

An in-vivo indwelling stent is used for treating various diseases caused by stenosis or occlusion of a blood vessel or other lumens in a living body. The stent is formed in a tubular shape to expand the stenotic site or the occluded site and ensure the lumen thereof.

Since the stent is inserted into the body from outside the body, the stent has a small diameter when inserted, expands and increases in diameter at a target stenotic or occluded site, and maintains the lumen at the site.

Typically, stents are cylindrical members obtained by processing metallic wires or metallic tubes. A stent is mounted to a catheter or the like in a radially contracted state, is inserted into a living body, and is expanded at a target site by some method to be fixed in close contact with the inner wall of the lumen at the site, thereby maintaining the shape of the lumen.

Stents are classified into a self-expandable stent and a balloon-expandable stent depending on a function and a placement method. A balloon-expandable stent that does not have a self-expanding function is mounted on a balloon. The balloon-expandable stent is inserted into a target site, and then, the balloon is dilated to expand (plastically deform) the stent by the expansion force of the balloon, whereby the stent is fixed in close contact with the inner surface of the target lumen. This type of stent requires the expansion of the stent as described above. On the other hand, a self-expandable stent has a self-expanding function. The self-expandable stent is inserted into a living body in a radially contracted state, and is deployed to open and is restored to its originally expanded state at a target site. Thus, the stent is fixed in close contact with the inner wall of the lumen and maintains the shape of the lumen at the target site.

One of the causes of ischemic cerebral artery disorders is stenosis/occlusion of the intracranial artery. As a general treatment method, antiplatelet therapy can be used to reduce risks. However, there is a limit in medical therapy, and a patient with drug resistance is treated by balloon dilation or stent placement. Stent placement technique has a problem that there are many perioperative complications, and one of the factors of such complications is formation of a thrombus in a stent. The brain is said to have poor medication accessibility due to the blood-brain barrier, and it is considered that the brain has an environment in which complications due to thrombus formation in the stent are likely to occur even if antithrombotic therapy is continued after surgery.

The intracranial blood vessels are often intricately tortuous. When a stent having low trackability is placed at a tortuous portion, buckling occurs such that a stent strut enters the lumen of the blood vessel to hamper blood flow. Thus, a thrombus is generated. In addition, the stent strut may damage the blood vessel wall and cause perforation or injury. In order to reduce a risk of formation of a thrombus in a stent, the stent needs to be designed to have excellent trackability without damaging blood vessels.

As a self-expandable stent, a stent disclosed in JP 2003-93519 A (US 2002/0193868 A, US 2004/0138730 A, US 2006/0149355 A, EP 1266640 A) has been proposed.

The stent disclosed in JP 2003-93519 A (US 2002/0193868 A, US 2004/0138730 A, US 2006/0149355 A, EP 1266640 A) includes a plurality of wavy struts extending in the axial direction from one end to the other end of the stent and arranged in the circumferential direction of the stent, and a plurality of connection struts connecting adjacent wavy struts of the plurality of wavy struts and extending in a predetermined long axis direction, wherein an end of each of the wavy struts is coupled to an end of the adjacent wavy strut.

In addition, an applicant of the present application proposes a stent disclosed in WO 2010/035721 A (US 2011/0196475 A, U.S. Pat. No. 8,518,102, EP 2322121 A).

The stent disclosed in WO 2010/035721 A (US 2011/0196475 A, U.S. Pat. No. 8,518,102, EP 2322121 A) includes a plurality of first wavy struts (3) extending in the axial direction of the stent (1) and arranged in the circumferential direction of the stent, a plurality of second wavy struts (4) each of which is arranged between the first wavy struts (3), and one or a plurality of connection struts (5) each of which connects the first wavy strut (3) and the second wavy strut (4) adjacent to each other, and extends in a predetermined long axis direction. The apex (41, 42) of the second wavy strut (4) is shifted by a predetermined length in the axial direction of the stent with respect to the apex (31, 32) of the first wavy strut (3), the apex (31, 32) being close to the apex (41, 42) in the circumferential direction of the stent (1) and being curved in the same direction.

### SUMMARY

The stent disclosed in JP 2003-93519 A (US 2002/0193868 A, US 2004/0138730 A, US 2006/0149355 A, EP 1266640 A) includes a plurality of wavy struts extending in the axial direction from one end to the other end of the stent and arranged in the circumferential direction of the stent. The present inventors have found as a result of research that the stent disclosed in JP 2003-93519 A (US 2002/0193868 A, US 2004/0138730 A, US 2006/0149355 A, EP 1266640 A) has insufficient expansion force and trackability because the wavy strut extends in the axial direction.

The stent disclosed in WO 2010/035721 A (US 2011/0196475 A, U.S. Pat. No. 8,518,102, EP 2322121 A) has a certain degree of trackability, but is desired to have higher trackability.

In view of this, an in-vivo indwelling stent and a stent delivery system are disclosed here that have excellent retractability by radial compression, blood vessel trackability after being placed in a blood vessel, and satisfactory vasodilating effect.

An aspect of the disclosure here involves the following.

An in-vivo indwelling stent extends in an axial direction, has a central portion in the axial direction and is expandable from a contracted state to an expanded state. The stent comprises a plurality of annular bodies each of which is constituted by a linear body that is in an annular shape, and connection portions that connect the annular bodies that are adjacent to each other, wherein
a plurality of the annular bodies located on at least the central part of the stent includes a first pattern annular body and a second pattern annular body that are alternately arranged in an axial direction of the stent,
the first pattern annular body has an endless zigzag linear shape including a plurality of one-end-side apexes, a plurality of other-end-side apexes, and a plurality of curved linear bodies that connect the plurality of one-end-side apexes and the plurality of other-end-side apexes and that protrude in a first circumferential direction,
the second pattern annular body has an endless zigzag linear shape including a plurality of one-end-side apexes, a plurality of other-end-side apexes, and a plurality of curved linear bodies that connect the plurality of one-end-side apexes and the plurality of other-end-side apexes and that protrude in a second circumferential direction opposite to the first circumferential direction, and
80% or more of the one-end-side apexes and the other-end-side apexes of the annular bodies that are adjacent to each other are connected by the connection portions.

Furthermore, another aspect of the disclosure involves the following.

An in-vivo indwelling stent extending in an axial direction and having a substantially tubular shape, the stent comprising:
a plurality of wavy linear bodies extending in an axial direction from one end side to another end side and arranged in a circumferential direction of the stent; and
a plurality of connection portions connecting the wavy linear bodies adjacent to each other, the plurality of connection portions extending in a predetermined long axis direction, wherein
the wavy linear bodies include a plurality of first linear curved portions whose central parts protrude in a first circumferential direction of the stent and a plurality of second linear curved portions whose central parts protrude in a second circumferential direction opposite to the first circumferential direction, the first linear curved portions and the second linear curved portions being alternately arranged in the axial direction of the stent,
the connection portions include a first pattern curved connection portion located between the first linear curved portions of the wavy linear bodies that are adjacent to each other in the circumferential direction, the first pattern curved connection portion connecting a portion on the one end side with respect to the central part of the first linear curved portion that is one of the first linear curved portions and that is located on the first circumferential direction side, and a portion near the central part and on the other end side of the other first linear curved portion, and a second pattern curved connection portion located between the second linear curved portions of the wavy linear bodies that are adjacent to each other in the circumferential direction, the second pattern curved connection portion connecting a portion on the one end side with respect to the central part of the second linear curved portion that is one of the second linear curved portions and that is located on the second circumferential direction side, and a portion near the central part and on the other end side of the other second linear curved portion,
a plurality of the first pattern curved connection portions is arranged in the circumferential direction of the stent with central parts protruding in the first circumferential direction, and a plurality of the second pattern curved connection portions is arranged in the circumferential direction of the stent with central parts protruding in the second circumferential direction.

A still further aspect of the disclosure here involves the following.

An in-vivo indwelling stent extending in an axial direction and having a substantially tubular shape comprises:
A plurality of first pattern annular portions in which a plurality of S-shaped curved linear bodies extending in the axial direction of the stent is arranged in a circumferential direction; and
a plurality of second pattern annular portions in which a plurality of inverted S-shaped curved linear bodies extending in the axial direction of the stent is arranged in the circumferential direction, the first pattern annular portions and the second pattern annular portions being alternately arranged in the axial direction of the stent, wherein
a first pattern annular portion and a second pattern annular portion adjacent to each other in the axial direction among the first pattern annular portions and the second pattern annular portions include an overlapping portion in which an end of the S-shaped curved linear body and an end of the inverted S-shaped curved linear body overlap each other in the circumferential direction between ends of the first pattern annular portion and the second pattern annular portion, the first pattern annular portion and the second pattern annular portion adjacent to each other in the axial direction being connected to each other in the overlapping portion, and
in the overlapping portion, one end of the inverted S-shaped curved linear body is connected to a portion located on one end side with a predetermined length from the other end of the S-shaped curved linear body adjacent to the inverted S-shaped curved linear body on one end side in the axial direction of the stent, the other end of the inverted S-shaped curved linear body is connected to a portion located on the other end side with a predetermined length from one end of the S-shaped curved linear body adjacent to the inverted S-shaped curved linear body on the other end side in the axial direction of the stent, one end of the S-shaped curved linear body is connected to a portion located on one end side with a predetermined length from the other end of the inverted S-shaped curved linear body adjacent to the S-shaped curved linear body on one end side in the axial direction of the stent, and the other end of the S-shaped curved linear body is connected to a portion located on the other end side with a predetermined length from one end of the inverted S-shaped curved linear body adjacent to the S-shaped curved linear body on the other end side in the axial direction of the stent.

An additional aspect of the disclosure here involves the following.

A stent delivery system comprising: a sheath; the stent accommodated in a distal portion of the sheath; and a shaft that is slidably inserted through the sheath for releasing the stent from a distal end of the sheath, wherein the stent is formed into a substantially cylindrical shape, is compressed in a central axis direction when inserted in a living body, and expands outward to be restored to the shape before compression when placed in the living body.

### BRIEF DESCRIPTION OF THE DRAWINGS

FIG. 1 is a front view of an in-vivo indwelling stent according to one embodiment when expanded.

FIG. 2 is a developed view of the in-vivo indwelling stent of FIG. 1.

FIG. 3 is a partially enlarged view of FIG. 2.

FIG. 4 is a developed view of the in-vivo indwelling stent of FIG. 1 when contracted.

FIG. 5 is a front view of an in-vivo indwelling stent according to another embodiment when expanded.

FIG. 6 is an explanatory diagram using a developed view of the in-vivo indwelling stent of FIG. 5.

FIG. 7 is a front view of an in-vivo indwelling stent according to still another embodiment when expanded.

FIG. 8 is an explanatory diagram using a developed view of the in-vivo indwelling stent of FIG. 7.

FIG. 9 is a diagram illustrating a state in which the in-vivo indwelling stent is placed in a tube such that an outer portion presses an inner surface of the tube, and the tube is bent.

FIG. 10 is a partially enlarged explanatory diagram of the in-vivo indwelling stent illustrated in FIG. 1.

FIG. 11 is a partially omitted front view of a stent delivery system according to the embodiment of the present disclosure.

FIG. 12 is a partially cutaway enlarged view of a distal portion of the stent delivery system illustrated in FIG. 11.

### DETAILED DESCRIPTION

An in-vivo indwelling stent disclosed here will be described by way of embodiments illustrated in the drawings, with such embodiments representing examples of the new stent disclosed here.

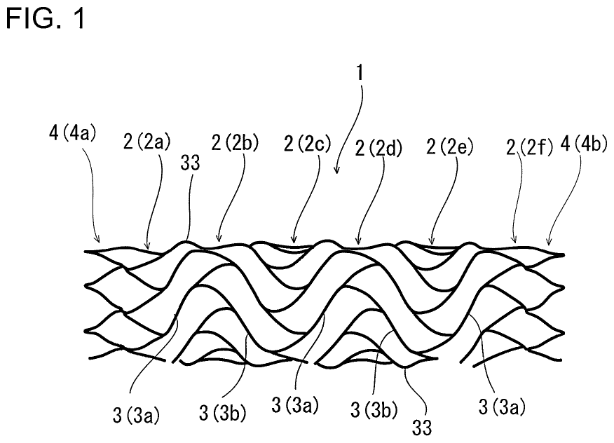

As illustrated in FIGS. 1 to 3, an in-vivo indwelling stent 1 according to the disclosure here includes a plurality of annular bodies 2 (2a, 2b, 2c, 2d, 2e, 2f) formed into an annular shape using linear bodies (struts) and arranged in an axial direction, and connection portions 3 (3a, 3b) that connect the adjacent annular bodies.

The plurality of annular bodies 2 (2a, 2b, 2c, 2d, 2e, 2f) located at least in the central part of the stent 1 includes first pattern annular bodies 2a, 2c, and 2e and second pattern annular bodies 2b, 2d, and 2f that are alternately arranged (alternately located) in the axial direction of the stent.

The first pattern annular bodies 2a, 2c, and 2e each have an endless zigzag linear shape having a plurality of one-end-side apexes 21a, a plurality of other-end-side apexes 22a, and a plurality of curved linear bodies (substantially arc-shaped linear bodies) 23a and 24a connecting the one-end-side apexes 21a and the other-end-side apexes 22a and protruding in a first circumferential direction. In FIGS. 1 to 3, the left side is the “one end side”, the right side is the “other end side”, and the downward direction is the “first circumferential direction”.

The second pattern annular bodies 2b, 2d, and 2f each have an endless zigzag linear shape having a plurality of one-end-side apexes 21b, a plurality of other-end-side apexes 22b, and a plurality of curved linear bodies (substantially arc-shaped linear bodies) 23b and 24b connecting the one-end-side apexes 21b and the other-end-side apexes 22b and protruding in a second circumferential direction opposite to the first circumferential direction. In this configuration, 80% or more (desirably 90% or more, particularly desirably 100%) of the one-end-side apexes 21a and 21b and the other-end-side apexes 22a and 22b of the adjacent annular bodies are connected by the connection portions 3 (3a, 3b).

The stent 1 can be effectively used as a stent for expansion of a cerebral artery (stent for treatment of cerebral artery stenosis).

The stent 1 according to the present embodiment is a so-called self-expandable stent that has a substantially cylindrical shape, is compressed in the central axis direction when inserted in a living body, and expands outward to be restored to the shape before compression when placed in the living body.

As illustrated in FIGS. 1 to 3, the stent 1 according to one aspect of the present disclosure includes the plurality of annular bodies 2 (2a, 2b, 2c, 2d, 2e, 2f) that is formed into an annular shape using linear bodies and that is arranged in the axial direction, and connection portions 3 (3a, 3b) that connect the adjacent annular bodies.

The number of the annular bodies 2 (excluding those at both ends) of the stent according to this embodiment is preferably 2 to 100, and particularly preferably 3 to 50. The number of the annular bodies 2 (excluding those at both ends) of the stent 1 is six.

The first pattern annular bodies 2a, 2c, and 2e and the second pattern annular bodies 2b, 2d, and 2f of the plurality of annular bodies 2 (2a, 2b, 2c, 2d, 2e, 2f) located at least in the central part of the stent 1, specifically, the plurality of annular bodies 2 (2a, 2b, 2c, 2d, 2e, 2f) excluding those at both ends of the stent 1, are alternately arranged in the axial direction of the stent.

The first pattern annular bodies 2a, 2c, and 2e each have an endless zigzag linear shape having the plurality of one-end-side apexes 21a, the plurality of other-end-side apexes 22a, and the plurality of curved linear bodies (in other words, substantially arc-shaped linear bodies) 23a and 24a connecting the one-end-side apexes 21a and the other-end-side apexes 22a and protruding in the first circumferential direction.

The second pattern annular bodies 2b, 2d, and 2f each have an endless zigzag linear shape having the plurality of one-end-side apexes 21b, the plurality of other-end-side apexes 22b, and the plurality of curved linear bodies (in other words, substantially arc-shaped linear bodies) 23b and 24b connecting the one-end-side apexes 21b and the other-end-side apexes 22b and protruding in the second circumferential direction opposite to the first circumferential direction.

The curved linear bodies of the first pattern annular bodies 2a, 2c, and 2e and the curved linear bodies of the second pattern annular bodies 2b, 2d, and 2f each have a substantially arc shape. Specifically, the curved linear bodies of the first pattern annular bodies 2a, 2c, and 2e each have both ends and a curved-portion apex that is located between both ends and that extends in the first circumferential direction of the stent 1. The curved linear bodies of the second pattern annular bodies 2b, 2d, and 2f each have both ends and a curved-portion apex that is located between both ends and that extends in the second circumferential direction of the stent 1 that is opposite to the first circumferential direction.

The stent 1 according to the present embodiment has at least two first pattern annular bodies, and the number of second pattern annular bodies is N−1 or more and N+1 or less when the number of first pattern annular bodies is N. Specifically, the number of the first pattern annular bodies is preferably 2 to 50, and particularly preferably 2 to 25. The number of second pattern annular bodies is ±1 of the number of first pattern annular bodies.

80% or more of the one-end-side apexes 21a and 21b and the other-end-side apexes 22a and 22b of the adjacent annular bodies are connected by the connection portions 3 (3a, 3b). Preferably, 90% or more, particularly preferably 100% of the one-end-side apexes 21a and 21b and the other-end-side apexes 22a and 22b of the adjacent annular bodies are connected by the connection portions 3 (3a, 3b).

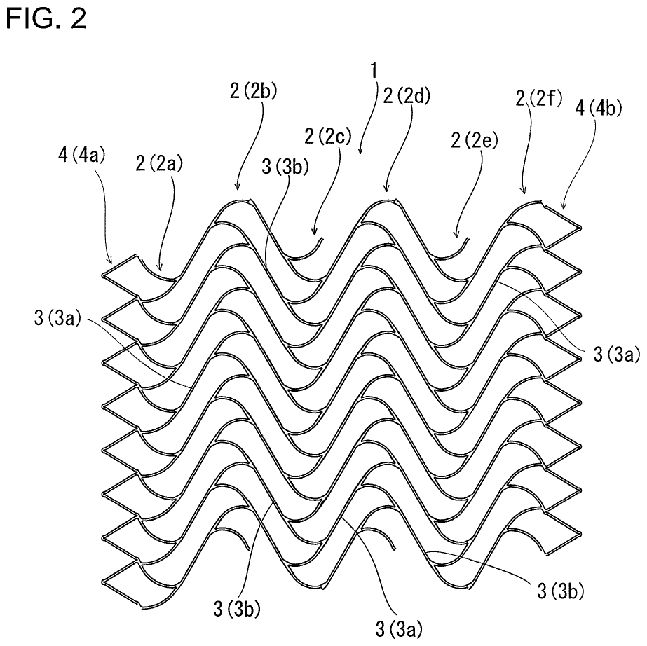

In this stent 1, all the apexes of the annular bodies of the stent are connected to the connection portions or the adjacent annular bodies, except for the apexes located at both axial ends (i.e., the left and right ends in FIG. 2). In particular, in the present embodiment, all the one-end-side apexes 21a and 21b and the other-end-side apexes 22a and 22b are connected by the connection portions 3 (3a, 3b) except for the apexes located at both axial ends.

In addition, in the stent 1, the apex of each of the annular bodies and the corresponding connection portion have continuity, and are continuously connected without having a distinct bent portion. Specifically, the connection portions 3 (3a, 3b) each have a first outer edge 28 and a second outer edge 31 that are substantially linear. The apexes (for example, 21b or 22b) of the annular bodies each have plural outer edges (29, 32), at least some of which are curved, that are gently connected to the end of the respective connection portion 3 (3a, 3b) without having a distinct bent portion.

As illustrated in FIG. 10, the apex (for example, 21b or 22b) of each of the annular bodies has a first outer edge 29 on the first circumferential direction side (lower side in FIG. 10) and a second outer edge 32 on the second circumferential direction side (upper side in FIG. 10). The first outer edge 28 of the connection portions 3 and the first outer edge 29 of the apexes of the annular bodies are contiguous along a bifurcated part on the first circumferential direction side (lower side in FIG. 10). Similarly, the second outer edge 31 of the connection portions 3 and the second outer edge 32 of the apexes of the annular bodies are contiguous along a bifurcated part on the second circumferential direction side (upper side in FIG. 10).

The second outer edge 31 and the second outer edge 32 on the second circumferential direction side are gently contiguous. The second outer edge 31 and the second outer edge 32 on the second circumferential direction side preferably have any one of a configuration in which they have so-called tangency continuity (G1 continuity) in which the tangential directions coincide with each other, a configuration in which they are curved in the same direction with substantially the same curvature, and a configuration in which they have tangency continuity (G1 continuity) and also have curvature continuity in which the curvatures at the contact point of the outer edges 31 and 32 also coincide with each other.

Similarly, the first outer edge 28 and the first outer edge 29 on the first circumferential direction side are gently contiguous without having a distinct bent portion. The first outer edge 28 and the first outer edge 29 on the first circumferential direction side preferably have any one of a configuration in which they have so-called tangency continuity (G1 continuity) in which the tangential directions coincide with each other, a configuration in which they are curved in the same direction with substantially the same curvature, and a configuration in which they have tangency continuity (G1 continuity) and also have curvature continuity in which the curvatures at the contact point of the first outer edge 28 and the first outer edge 29 also coincide with each other.

In particular, in the present embodiment, the second outer edge 31 and the second outer edge 32 on the second circumferential direction side have substantially tangency continuity (G1 continuity), and also have curvature continuity in which the curvatures of the outer edge 31 and the outer edge 32 at the contact point coincide with each other. In addition, the first outer edge 28 and the first outer edge 29 on the first circumferential direction side have substantially tangency continuity (G1 continuity), and also have curvature continuity in which the curvatures of the outer edge 28 and the outer edge 29 at the contact point coincide with each other.

In addition, as illustrated in FIG. 10, the stent 1 according to the present embodiment does not have a region where radiuses of circles in contact with the linear bodies (struts) on the second circumferential direction side (upper side in FIG. 10) and on the first circumferential direction side (lower side in FIG. 10) in the vicinity of the connection part between the apex of the annular body and the connection portion are equal to or less than a predetermined value. Specifically, there is no region where the radius of a circle in contact with the linear body in the vicinity of the connection part between the apex of the annular body and the connection portion is equal to or less than 0.01 mm. In addition, it is preferable that there is no region where the radiuses of circles in contact with the linear bodies (struts) on the second circumferential direction side and on the first circumferential direction side in the vicinity of the connection part between the apex of the annular body and the connection portion are equal to or less than 0.01 mm. In particular, it is preferable that there is no region where the radius of the circle in contact with the linear body (strut) is equal to or less than 0.008 mm.

In addition, in the stent 1 according to the present embodiment, in the vicinity of the connection part between the apex of the annular body and the connection portion, the centers of the circles in contact with the struts on the second circumferential direction side and on the first circumferential direction side are located in the direction opposite to the direction in which the curved linear body of the annular body protrudes as viewed from the contact point.

The connection portions 3a adjacent to each other in the circumferential direction of the stent 1 are inclined in the same direction, have substantially the same shape, and are substantially parallel. Similarly, the connection portions 3b adjacent to each other in the circumferential direction of the stent 1 are inclined in the same direction, have substantially the same shape, and are substantially parallel. The connection portion 3a and the connection portion 3b adjacent to each other in the axial direction of the stent are inclined in different directions. In the present embodiment, the connection portion 3a and the connection portion 3b adjacent to each other in the axial direction have an inverted shape (substantially symmetrical shape).

In the stent 1 according to the present embodiment, the connection portion 3a and the connection portion 3b are curved (slightly curved) in different directions at both ends to be connected to the ends of the annular bodies, and have straight portions at the central parts thereof. The connection portion 3a and the connection portion 3b may have substantially no linear element and may be curved as a whole.

In addition, the linear bodies (struts) constituting the annular bodies and the connection portions all have the same line width except for portions where the annular bodies branch.

The stent 1 according to the present embodiment includes a one-end annular body 4a located at one end and an other-end annular body 4b located at the other end. In the stent 1 according to the present embodiment, the one-end annular body 4a and the other-end annular body 4b are formed in a zigzag shape by straight linear portions. The one-end annular body 4a and the other-end annular body 4b may be constituted by curved linear bodies (in other words, substantially arc-shaped linear bodies) as in the above-described first pattern annular body or the second pattern annular body. Furthermore, the stent 1 according to the present embodiment does not have one-end-side apexes that are free ends except for the one-end annular body 4a. Therefore, when the stent that is accommodated in a sheath with the other end side being the distal side is inserted into a blood vessel, and then, exposed to some extent, the stent can be accommodated in the sheath again. In addition, the stent 1 according to the present embodiment does not have other-end-side apexes that are free ends except for the other-end annular body 4b. Therefore, when the stent that is accommodated in a sheath with the one end side being the distal side is inserted into a blood vessel, and then, exposed to some extent, the stent can be accommodated in the sheath again.

In addition, in the stent 1 according to the present embodiment, the lengths of the annular bodies 2 (2a, 2b, 2c, 2d, 2e, 2f) in the axial direction of the stent are the same as or slightly longer than the lengths of the connection portions 3 (3a, 3b) in the axial direction when the stent 1 is expanded. The lengths of the annular bodies 2 (2a, 2b, 2c, 2d, 2e, 2f) in the axial direction of the stent when the stent 1 is expanded are preferably 0.2 mm to 20 mm, and particularly preferably 0.5 mm to 10 mm. The lengths of the connection portions 3 (3a, 3b) in the axial direction of the stent when the stent 1 is expanded are preferably 0.05 mm to 20 mm, and particularly preferably 0.1 mm to 10 mm. The distance between the connection portions 3 (3a, 3b) in the circumferential direction when the stent 1 is expanded is preferably 0.1 mm to 10 mm, and particularly preferably 0.2 mm to 5 mm. The inclination angle (inclination angle of a straight line connecting one end and the other end of the connection portion 3a, 3b with respect to the central axis of the stent) of the connection portion 3 (3a, 3b) with respect to the central axis of the stent is preferably 1 degree to 89 degrees, and particularly preferably 50 degrees to 70 degrees.

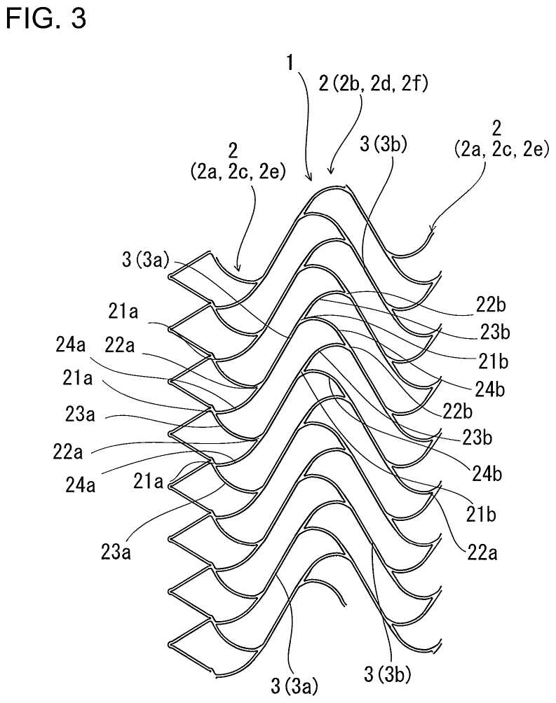

In the stent 1 according to the present embodiment, the connection portions include a first pattern connection portion 3a and a second pattern connection portion 3b as illustrated in FIGS. 2 and 3. In the first pattern annular body 2 (2a, 2c, 2e), two curved linear bodies 23a and 24a extend from the one-end-side apex 21a to two different other-end-side apexes 22a. In the second pattern annular body 2 (2b, 2d, 2f), two curved linear bodies 23b and 24b extend from the one-end-side apex 21b to two different other-end-side apexes 22b. The first pattern connection portion 3a extends to the other end side and in the second circumferential direction, and connects the other-end-side apexes 22a of the first pattern annular body 2 (2a, 2c, 2e) and the one-end-side apexes 21b of the second pattern annular body 2 (2b, 2d, 2f). The second pattern connection portion 3b extends to the other end side and in the first circumferential direction, and connects the other-end-side apexes 22b of the second pattern annular body 2 (2b, 2d, 2f) and the one-end-side apexes 21a of the first pattern annular body 2 (2a, 2c, 2e). Therefore, the stent 1 has excellent retractability by radial compression, blood vessel trackability after being placed in a blood vessel, and satisfactory vasodilating effect.

In addition, in the stent 1 according to the present embodiment, the linear bodies (struts) constituting the annular bodies each have a curved shape (substantially arc shape), so that the struts constituting annular portions gradually become parallel to the axial direction while being deformed as a whole by a force received from the connection portions in a state where the stent is contracted to be housed in the sheath. Therefore, it is possible to suppress local deformation generated in the stent strut at the time of contraction and to smoothly move (reduce the diameter) the stent.

In addition, since the apexes of the annular bodies and the connection portions are continuously connected, stress generated when the stent is deformed (for example, increased or decreased in diameter, or bent) is dispersed, and specific deformation hardly occurs. Therefore, it is possible to suppress the stent from protruding from the cylindrical side surface and impairing the effect of the stent of improving the blood flow. In other words, it is possible to suppress local application of stress on the portions where the apexes of the annular bodies and the connection portions are connected, when the stent is placed in a tortuous blood vessel.

In addition, the stent 1 includes a both-side curved closed linear portion of a first pattern having substantially a rectangular shape and extending in a second direction of the stent with substantially the same width with both side ends being tapered by the apexes 21a and 22b, and a both-side curved closed linear portion of a second pattern having substantially a rectangular shape and extending in a first direction of the stent with substantially the same width with both side ends being tapered by the apexes 21b and 22a. A plurality of the both-side curved closed linear portions of the first pattern having substantially a rectangular shape is arranged in parallel in the circumferential direction of the stent, and a plurality of the both-side curved closed linear portions of the second pattern having substantially a rectangular shape is similarly arranged in parallel in the circumferential direction of the stent. One end of the both-side curved closed linear portion of the second pattern having substantially a rectangular shape enters between other ends of the both-side curved closed linear portions of the first pattern having substantially a rectangular shape, and one end of the both-side curved closed linear portion of the first pattern having substantially a rectangular shape enters between the other ends of the both-side curved closed linear portions of the second pattern having substantially a rectangular shape. Except for both ends of the stent 1, the stent 1 is formed by interconnecting the both-side curved closed linear portions of the first pattern having substantially a rectangular shape and the both-side curved closed linear portions of the second pattern having substantially a rectangular shape. The both-side curved closed linear portions of the first pattern having substantially a rectangular shape and the both-side curved closed linear portions of the second pattern having substantially a rectangular shape are alternately arranged in the axial direction.

It is considered that this configuration can also contribute to excellent retractability by radial compression, blood vessel trackability after being placed in a blood vessel, and satisfactory vasodilating effect of the stent 1.

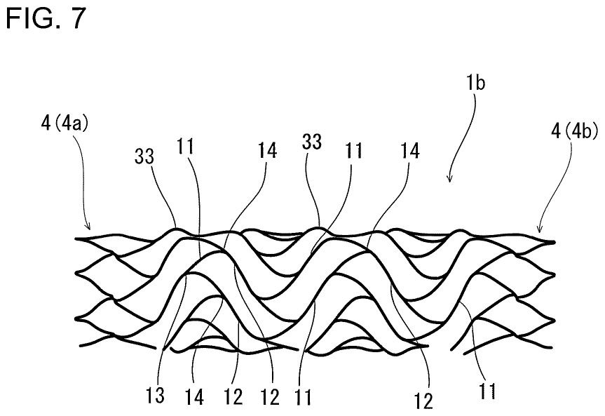

In addition, in the stent 1 according to the present embodiment, central parts 33 of the connection portions 3 (3a, 3b) are curved and protrude to the outside of the stent 1 during expansion, such as shown by way of example in FIGS. 5 and 7. In other words, the central parts 33 of the connection portions 3 (3a, 3b) are in a curved state protruding to the outside of the stent at the time of expansion. Therefore, in the expanded state of the stent, the stent 1 has protrusions each of which is located between the adjacent annular bodies and protrudes to the outside of the stent. The protrusion is formed by the intermediate portion of the connection portion protruding outward as described above.

In addition, an amount of protrusion gradually increases from both ends to the central part of the connection portion, and the central part is a vertex of the protrusion. When a region between the protrusions is regarded as a recess, the stent 1 has protruding and recessed sections on the outer side, and a cycle of the protruding and recessed sections in the axial direction is a distance in the axial direction between the one-end-side apexes of the adjacent annular bodies or between the other-end-side apexes of the adjacent annular bodies. For this reason, the contact with a part of the living body into which the stent is inserted is improved, and the movement of the stent is satisfactorily regulated.

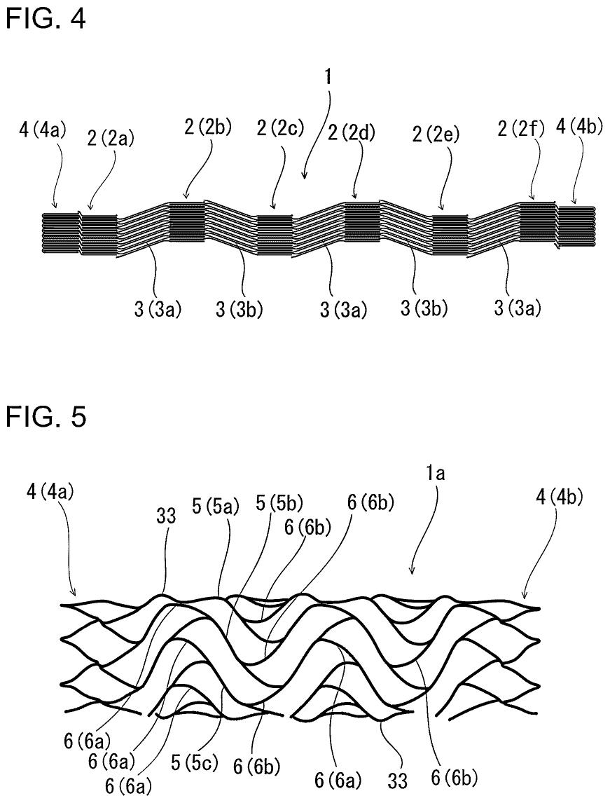

In addition, the stent 1 according to the present embodiment is in a state as illustrated in FIG. 4 when contracted (when decreased in diameter). That is, the stent 1 does not have a portion that inhibits a decrease in diameter of other portions when decreased in diameter, and thus has excellent deformability in the radial direction (is satisfactorily decreased in diameter).

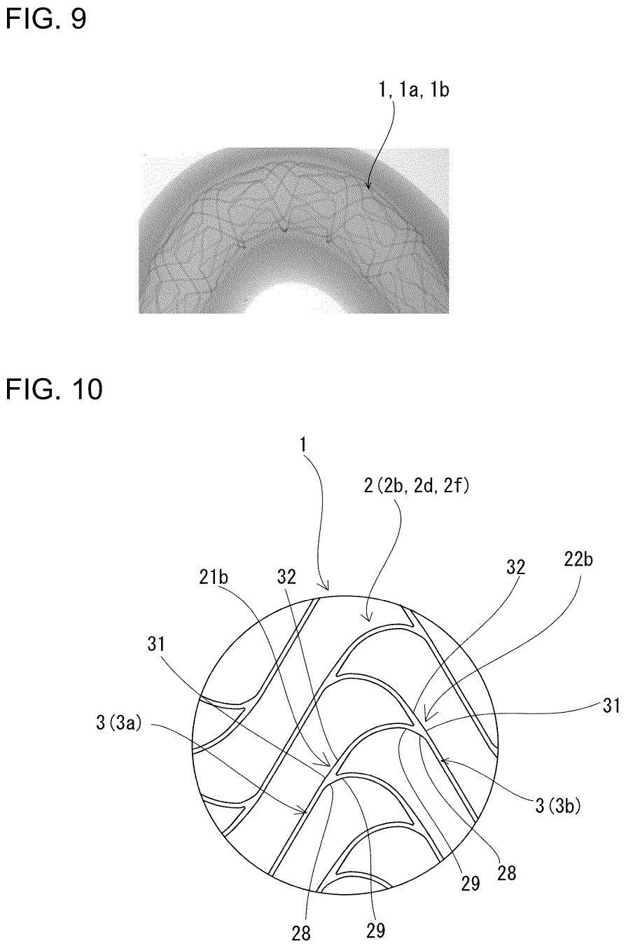

Further, when the stent 1 according to the present embodiment is placed in a tube and the tube is largely bent, the stent is satisfactorily bent and deformed without being buckled and prevents buckling of the tube to maintain the lumen and the bent state, as illustrated in FIG. 9.

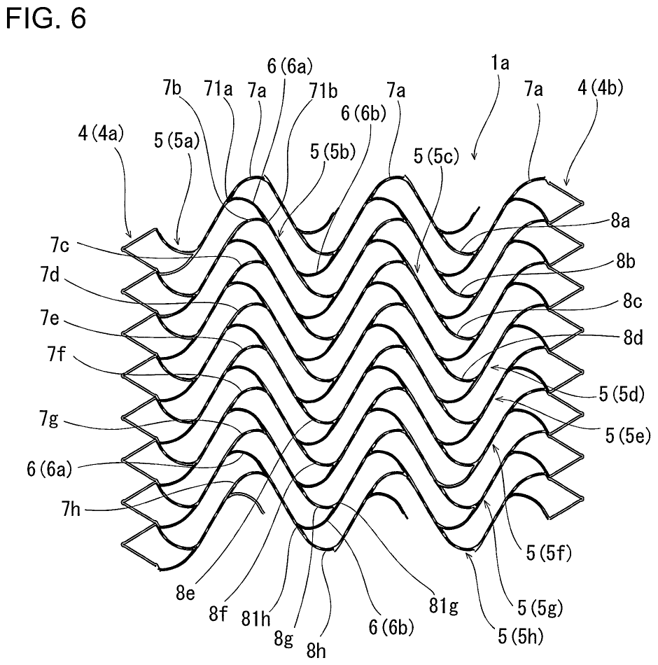

In addition, as illustrated in FIGS. 5 and 6, the stent according to the disclosure here is an in-vivo indwelling stent 1a formed in a substantially tubular shape and including a plurality of wavy linear bodies 5 (5a, 5b, 5c, 5d, 5e, 5f, 5g, 5h) extending in the axial direction from one end to the other end and arranged in the circumferential direction of the stent, and a plurality of connection portions 6 (6a, 6b) that connects the wavy linear bodies 5 (5a, 5b, 5c, 5d, 5e, 5f, 5g, 5h) adjacent in the circumferential direction and that extends in a predetermined long axis direction.

The wavy linear bodies 5 (5a, 5b, 5c, 5d, 5e, 5f, 5g, 5h) include a plurality of first linear curved portions 7 (7a, 7b, 7c, 7d, 7e, 7f, 7g, 7h) each having a central part protruding in a first circumferential direction (upward direction in FIG. 6) of the stent and a plurality of second linear curved portions 8 (8a, 8b, 8c, 8d, 8e, 8f, 8g, 8h) each having a central part protruding in a second circumferential direction opposite to the first circumferential direction, the first linear curved portions 7 and the second linear curved portions 8 being alternately arranged in the axial direction of the stent.

The connection portion 6 (6a, 6b) includes: a first pattern curved connection portion 6a that is located between the first linear curved portions (for example, 7a and 7b) of the wavy linear bodies 5 (5a, 5b, 5c, 5d, 5e, 5f, 5g, 5h) adjacent to each other in the circumferential direction and connects a portion 71a located on one end side with respect to the central part of the first linear curved portion 7a (for example) that is one of the first linear curved portions and that is located on the first circumferential direction side and a portion 71b located on the other end side and near the central part of the other first linear curved portion 7b (for example); and a second pattern curved connection portion 6b that is located between the second linear curved portions (for example, 8h and 8g) of the wavy linear bodies 5 (5a, 5b, 5c, 5d, 5e, 5f, 5g, 5h) adjacent to each other in the circumferential direction and connects a portion 81h located on one end side with respect to the central part of the second linear curved portion 8h (for example) that is one of the second linear curved portions and that is located on the second circumferential direction side and a portion 81g located on the other end side and near the central part of the other second linear curved portion 8g.

A plurality of the first pattern curved connection portions 6a is arranged in the circumferential direction of the stent with the central parts protruding in the first circumferential direction, and a plurality of the second pattern curved connection portions 6b is arranged in the circumferential direction of the stent with the central parts protruding in the second circumferential direction.

As indicated by a thick broken line in FIG. 6, the wavy linear bodies 5 (5a, 5b, 5c, 5d, 5e, 5f, 5g, 5h) are spaced at substantially equal intervals and arranged in the circumferential direction substantially in parallel, and a tubular skeleton of the stent 1a is constituted by a plurality of wavy linear bodies.

The stent preferably includes four or more wavy linear bodies. The number of wavy linear bodies is preferably 4 to 20, and particularly preferably 5 to 15. The stent 1a according to the present embodiment has eight wavy linear bodies. The distance between the wavy linear bodies 5 (5a, 5b, 5c, 5d, 5e, 5f, 5g, 5h) when the stent 1 is expanded is preferably 0.1 mm to 10 mm, and particularly preferably 0.2 mm to 5 mm.

In addition, as indicated by the thick broken line in FIG. 6, the wavy linear bodies 5 (5a, 5b, 5c, 5d, 5e, 5f, 5g, 5h) include a plurality of the first linear curved portions 7 (7a, 7b, 7c, 7d, 7e, 7f, 7g, 7h) each having a central part protruding in the first circumferential direction and a plurality of the second linear curved portions 8 (8a, 8b, 8c, 8d, 8e, 8f, 8g, 8h) each having a central part protruding in the second circumferential direction opposite to the first circumferential direction, the first linear curved portions 7 and the second linear curved portions 8 being alternately arranged. The wavy linear bodies 5 (5a, 5b, 5c, 5d, 5e, 5f, 5g, 5h) preferably include at least two of the first linear curved portions 7 (7a, 7b, 7c, 7d, 7e, 7f, 7g, 7h) and second linear curved portions 8 (8a, 8b, 8c, 8d, 8e, 8f, 8g, 8h).

The number of the first linear curved portions is preferably 1 to 50, and particularly preferably 2 to 25. The number of the second linear curved portions is preferably 1 to 50, and particularly preferably 2 to 25. In the stent 1a according to the present embodiment, the number of the first linear curved portions and the number of the second linear curved portions in the axial direction are each three, and the number of the first linear curved portions and the number of the second linear curved portions in the circumferential direction are each eight. In addition, in the stent 1a according to the present embodiment, the wavy linear bodies 5 (5a, 5b, 5c, 5d, 5e, 5f, 5g, 5h) are sinusoidal. In the stent 1a according to the present embodiment, the wavy linear bodies 5 (5a, 5b, 5c, 5d, 5e, 5f, 5g, 5h) have straight portions, and thus are not perfectly sinusoidal. However, the wavy linear bodies may be perfectly sinusoidal.

The length in the axial direction of the stent between the first linear curved portion and the second linear curved portion during expansion of the stent is preferably 0.1 mm to 10 mm, and particularly preferably 0.2 mm to 5 mm. In addition, the length in the circumferential direction of the stent between the first linear curved portion and the second linear curved portion during expansion of the stent is preferably 0.1 mm to 10 mm, and particularly preferably 0.2 mm to 5 mm. The inclination angle of the linear portion (straight line connecting ends of the linear portion) between the first linear curved portion and the second linear curved portion with respect to the central axis during the expansion of the stent is preferably 1 degree to 89 degrees, and particularly preferably 50 degrees to 70 degrees.

The stent 1a according to the present embodiment includes a plurality of the first pattern curved connection portions 6a and a plurality of the second pattern curved connection portions 6b indicated by a thick solid line in FIG. 6. In particular, in the stent 1a according to the present embodiment illustrated in FIG. 6, the first pattern curved connection portions 6a and the second pattern curved connection portions 6b each have a substantially arc shape.

The plurality of first pattern curved connection portions 6a is arranged in parallel in the circumferential direction of the stent with the vertexes of the curved portions protruding in the first circumferential direction as indicated by the thick solid line in FIG. 6. The plurality of second pattern curved connection portions 6b is arranged in parallel in the circumferential direction of the stent with the vertexes of the curved portions protruding in the second circumferential direction as indicated by the thick solid line in FIG. 6. The length in the axial direction of the stent between the first pattern curved connection portion 6a and the second pattern curved connection portion 6b is substantially the same as the length in the axial direction of the stent between the first linear curved portion and the second linear curved portion, and the length in the circumferential direction of the stent between the first pattern curved connection portion 6a and the second pattern curved connection portion 6b is substantially the same as the length in the circumferential direction of the stent between the first linear curved portion and the second linear curved portion. In particular, in the stent according to the present embodiment, the wavy linear bodies 5 (5a, 5b, 5c, 5d, 5e, 5f, 5g, 5h) are parallel and spaced from each other at equal intervals, and the spaced linear bodies are connected by the first pattern curved connection portions 6a and the second pattern curved connection portions 6b that are parallel and spaced from each other at equal intervals. Therefore, the stent 1a has excellent retractability by radial compression, blood vessel trackability after being placed in a blood vessel, and satisfactory vasodilating effect.

In the stent 1a, the first pattern curved connection portions 6a are provided in all the regions between the first linear curved portions (7a and 7b, for example) and the second pattern curved connection portions 6b are provided in all the regions between the second linear curved portions (8h and 8g, for example).

In addition, in the stent 1a according to the present embodiment, a central part 33 between the vertexes of the curved portions is also curved and protrudes to the outside of the stent 1a during expansion. Therefore, in the expanded state of the stent, the stent has protrusions that protrude to the outside of the stent. Due to the protrusions, the contact with a part of the living body into which the stent is inserted is improved, and the movement of the stent is satisfactorily regulated.

In addition, the stent 1a according to the present embodiment is also in a state as illustrated in FIG. 4 when contracted (when decreased in diameter). That is, the stent 1a does not ha

ve a portion that inhibits a decrease in diameter of other portions when decreased in diameter, and thus has excellent deformability in the radial direction (is satisfactorily decreased in diameter).

In addition, the stent 1a can also be effectively used as a stent for expansion of a cerebral artery (stent for treatment of cerebral artery stenosis). The stent 1a according to the present embodiment is a so-called self-expandable stent that has a substantially cylindrical shape, is compressed in the central axis direction when inserted in a living body, and expands outward to be restored to the shape before compression when placed in the living body.

Further, when the stent 1a according to the present embodiment is placed in a tube and the tube is largely bent, the stent is also satisfactorily bent and deformed without being buckled and prevents buckling of the tube to maintain the bent state, as illustrated in FIG. 9.

The stent 1a according to the present embodiment also includes a one-end annular body 4a located at one end and an other-end annular body 4b located at the other end. In the stent 1a according to the present embodiment, the one-end annular body 4a and the other-end annular body 4b are also formed in a zigzag shape by straight linear portions. The one-end annular body 4a and the other-end annular body 4b may be formed of curved linear bodies (in other words, substantially arc-shaped linear bodies) as in the first pattern annular body or the second pattern annular body of the stent 1 described above.

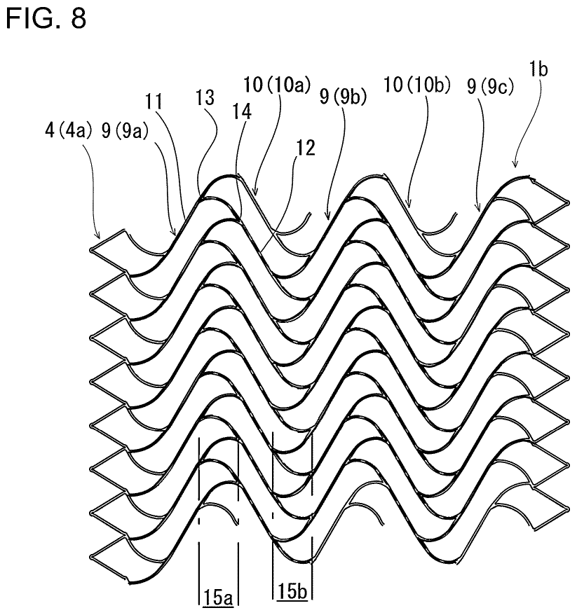

In addition, as illustrated in FIGS. 7 and 8, the stent according to the disclosure here is an in-vivo indwelling stent 1b formed in a substantially tubular shape and including: a plurality of first pattern annular portions 9 (9a, 9b, 9c) in which a plurality of S-shaped curved linear bodies 11 (indicated by a thick solid line in FIG. 8) extending in the axial direction of the stent is arranged in the circumferential direction; and a plurality of second pattern annular portions 10 (10a, 10b) in which a plurality of inverted S-shaped curved linear bodies 12 (indicated by a thick broken line in FIG. 8) extending in the axial direction of the stent is arranged in the circumferential direction, the first pattern annular portions 9 and the second pattern annular portions 10 being alternately arranged in the axial direction of the stent.

The first pattern annular portion 9 (9a, 9b, 9c) and the second pattern annular portion 10 (10a, 10b) adjacent to each other in the axial direction include an overlapping portion 15 (15a, 15b) in which an end of the S-shaped curved linear body (indicated by the thick solid line in FIG. 8) and an end of the inverted S-shaped curved linear body (indicated by the thick broken line in FIG. 8) overlap each other in the circumferential direction between the ends of both annular portions, and the first pattern annular portion and the second pattern annular portion adjacent to each other in the axial direction are connected to each other in the overlapping portion 15 (15a, 15b).

In the overlapping portion 15a, 15b, one end of the inverted S-shaped curved linear body 12 (indicated by the thick broken line in FIG. 8) is connected to a portion 13 located on one end side with a predetermined length from the other end of the S-shaped curved linear body 11 (indicated by the thick solid line in FIG. 8) adjacent to the inverted S-shaped curved linear body 12 on one end side in the axial direction of the stent, and the other end of the inverted S-shaped curved linear body 12 is connected to a portion located on the other end side with a predetermined length from one end of the S-shaped curved linear body 11 adjacent to the inverted S-shaped curved linear body 12 on the other end side in the axial direction of the stent. Further, in the overlapping portion 15, the other end of the S-shaped curved linear body 11 (indicated by the thick solid line in FIG. 8) is connected to a portion located on one end side with a predetermined length from the other end of the inverted S-shaped curved linear body 12 (indicated by the thick broken line in FIG. 8) adjacent to the S-shaped curved linear body 11 on one end side in the axial direction of the stent, and the other end of the S-shaped curved linear body 11 is connected to a portion 14 located on the other end side with a predetermined length from one end of the inverted S-shaped curved linear body 12 adjacent to the S-shaped curved linear body 11 on the other end side in the axial direction of the stent.

In the first pattern annular portion 9 (9a, 9b, 9c), a plurality of S-shaped curved linear bodies 11 (indicated by the thick solid line in FIG. 8) of the same shape are arranged in parallel in the circumferential direction at equal intervals. Similarly, in the second pattern annular portion 10 (10a, 10b), a plurality of inverted S-shaped curved linear bodies 12 (indicated by the thick broken line in FIG. 8) of the same shape are arranged in parallel in the circumferential direction at equal intervals. The S-shaped curved linear body 11 and the inverted S-shaped curved linear body 12 have shapes substantially symmetrical in the axial direction.

The first pattern annular portion 9 (9a, 9b, 9c) preferably includes four or more S-shaped curved linear bodies 11. The number of the S-shaped curved linear bodies 11 in the first pattern annular portion 9 is preferably 4 to 20, and particularly preferably 5 to 15.

The second pattern annular portion 10 (10a, 10b) preferably includes four or more inverted S-shaped curved linear bodies 12. The number of the inverted S-shaped curved linear bodies 12 in the second pattern annular portion 10 is preferably 4 to 20, and particularly preferably 5 to 15.

The number of S-shaped curved linear bodies 11 constituting the first pattern annular portion 9 (9a, 9b, 9c) and the number of inverted S-shaped curved linear bodies 12 constituting the second pattern annular portion 10 are the same.

The stent 1b according to the present embodiment has at least two first pattern annular portions, and the number of second pattern annular portions is N−1 or more and N+1 or less when the number of first pattern annular portions is N. In particular, the stent 1b preferably includes two or more of the first pattern annular portions 9 (9a, 9b, 9c) and the second pattern annular portions 10 (10a, 10b) in the axial direction.

Specifically, the number of the first pattern annular portions 9 (9a, 9b, 9c) in the stent 1b is preferably two or more, and particularly three or more. The stent 1b according to the present embodiment includes three first pattern annular portions 9. Further, the number of the second pattern annular portions 10 (10a, 10b) in the stent 1b is preferably one or more, and particularly preferably two or more. The stent 1b according to the present embodiment includes two second pattern annular portions 10.

The length of the S-shaped curved linear body 11 in the axial direction of the stent during expansion of the stent is preferably 0.45 mm to 60 mm, and particularly preferably 1.1 mm to 30 mm. In addition, the length of the S-shaped curved linear body 11 in the circumferential direction of the stent during expansion of the stent is preferably 0.1 mm to 20 mm, and particularly preferably 0.2 mm to 10 mm.

The length of the inverted S-shaped curved linear body 12 in the axial direction of the stent during expansion of the stent is preferably 0.45 mm to 60 mm, and particularly preferably 1.1 mm to 30 mm. In addition, the length of the inverted S-shaped curved linear body 12 in the circumferential direction of the stent during expansion of the stent is preferably 0.1 mm to 20 mm, and particularly preferably 0.2 mm to 10 mm.

In addition, the distance between the S-shaped curved linear bodies 11 during expansion of the stent is preferably 0.1 mm to 10 mm, and particularly preferably 0.2 mm to 5 mm.

The distance between the inverted S-shaped curved linear bodies 12 during expansion of the stent is preferably 0.1 mm to 10 mm, and particularly preferably 0.2 mm to 5 mm.

The inclination angles of the S-shaped curved linear body 11 and the inverted S-shaped curved linear body 12 with respect to the central axis during the expansion of the stent are preferably 1 degree to 89 degrees, and particularly preferably 50 degrees to 70 degrees.

The first pattern annular portion 9 (9a, 9b, 9c) and the second pattern annular portion 10 (10a, 10b) adjacent to each other in the axial direction include the overlapping portion 15 (15a, 15b) in which the end of the S-shaped curved linear body and the end of the inverted S-shaped curved linear body overlap each other in the circumferential direction between the ends of both annular portions. In the overlapping portion 15, the first pattern annular portion 9 (9a, 9b, 9c) and the second pattern annular portion 10 (10a, 10b) adjacent to each other in the axial direction are connected.

In the stent 1b according to the present embodiment, in regions between the S-shaped curved linear bodies 11 and the inverted S-shaped curved linear bodies 12 adjacent to each other in the axial direction, the ends of all the S-shaped curved linear bodies 11 located in the central part of the stent are connected to two inverted S-shaped curved linear bodies 12, and the ends of all the inverted S-shaped curved linear bodies 12 located in the central part of the stent are connected to two S-shaped curved linear bodies 11.

Since the overlapping portion 15a has the other end of the S-shaped curved linear body 11 and one end of the inverted S-shaped curved linear body 12, the number of linear bodies is larger than that of portions other than the overlapping portion, and thus, the overlapping portion 15a exhibits a strong expansion force. In addition, since the overlapping portion 15b has one end of the S-shaped curved linear body 11 and the other end of the inverted S-shaped curved linear body 12, the number of linear bodies is larger than that of portions other than the overlapping portion, and thus, the overlapping portion 15b exhibits a strong expansion force. As illustrated in FIG. 8, the overlapping portion 15a and the overlapping portion 15b have different shapes, and the shapes thereof are substantially symmetrical in the axial direction.

The length of the overlapping portion 15a, 15b in the axial direction of the stent during expansion of the stent is preferably 0.2 mm to 20 mm, and particularly preferably 0.5 mm to 10 mm.

In addition, in the stent 1b according to the present embodiment, a central part 33 between the vertexes of the curved portions is also curved and protrudes to the outside of the stent 1b during expansion. Therefore, in the expanded state of the stent, the stent has protrusions that protrude to the outside of the stent. Due to the protrusions, the contact with a part of the living body into which the stent is inserted is improved, and the movement of the stent is satisfactorily regulated.

In addition, the stent 1b according to the present embodiment is also in a state as illustrated in FIG. 4 when contracted (when decreased in diameter). That is, the stent 1b does not have a portion that inhibits a decrease in diameter of other portions when decreased in diameter, and thus has excellent deformability in the radial direction (is satisfactorily decreased in diameter).

In addition, the stent 1b can also be effectively used as a stent for expansion of a cerebral artery (stent for treatment of cerebral artery stenosis). The stent 1b according to the present embodiment is a so-called self-expandable stent that has a substantially cylindrical shape, is compressed in the central axis direction when inserted in a living body, and expands outward to be restored to the shape before compression when placed in the living body.

Further, when the stent 1b according to the present embodiment is placed in a tube and the tube is largely bent, the stent is also satisfactorily bent and deformed without being buckled and prevents buckling of the tube to maintain the bent state, as illustrated in FIG. 9.

The stent 1b according to the present embodiment also includes a one-end annular body 4a located at one end and an other-end annular body 4b located at the other end. In the stent 1b according to the present embodiment, the one-end annular body 4a and the other-end annular body 4b are also formed in a zigzag shape by straight linear portions. The one-end annular body 4a and the other-end annular body 4b may be formed of curved linear bodies (in other words, substantially arc-shaped linear bodies) as in the first pattern annular body or the second pattern annular body of the stent 1 described above.

The material constituting the stent is preferably a superelastic metal. As the superelastic metal, superelastic alloys are preferably used. The superelastic alloys herein are generally called shape memory alloys and exhibit superelasticity at least at a temperature of a living body (around 37° C.). Examples of superelastic alloys that are particularly preferably used include a superelastic alloy such as a Ti—Ni alloy containing 49 at % to 53 at % of Ni, a Cu—Zn alloy containing 38.5 wt % to 41.5 wt % of Zn, a Cu—Zn—X alloy (X=Be, Si, Sn, Al, Ga) containing 1 wt % to 10 wt % of X, a Ni—Al alloy containing 36 at % to 38 at % of Al, or a Mg—Sc alloy containing 15 at % to 25 at % of Sc. The Ti—Ni alloy is particularly preferable. In addition, the mechanical characteristics can be appropriately changed by using a Ti—Ni—X alloy (X=Co, Fe, Mn, Cr, V, Al, Nb, W, B, etc.) obtained by substituting a part of the Ti—Ni alloy with 0.01% to 10.0% of X, using a Ti—Ni—Y alloy (Y=Cu, Pb, Zr) obtained by substituting a part of the Ti—Ni alloy with 0.01% to 30.0% of Y, and selecting a cold working rate and/or the conditions of a final heat treatment. In addition, the mechanical characteristics can be appropriately changed by selecting the cold working rate and/or conditions of the final heat treatment using the Ti—Ni—X alloy. The buckling strength (yield stress when loaded) of the superelastic alloy to be used is 5 kg/mm² to 200 kg/mm² (22° C.), more preferably 8 kg/mm² to 150 kg/mm², and the restoring stress (yield stress when unloaded) is 3 kg/mm² to 180 kg/mm² (22° C.), more preferably 5 kg/mm² to 130 kg/mm². The superelasticity as used herein means a property of a metal such that, even when the metal is deformed (bent, extended, compressed) to an extent that ordinary metal is plastically deformed at use temperature, the deformed metal is restored substantially into its original shape before compression without requiring heating after the deformation is removed.

When the stent 1, 1a, 1b is used to, for example, dilate cerebral blood vessels, the diameter of the stent when the stent is expanded (when the stent is in an uncompressed state) is preferably about 0.5 mm to 6.0 mm, and particularly preferably 0.9 mm to 5.0 mm. In addition, the length of the stent when the stent is expanded (when the stent is in an uncompressed state) is preferably about 5 mm to 50 mm.

The thickness of the stent is preferably about 0.05 mm to 0.15 mm, and particularly preferably 0.06 mm to 0.13 mm. The width of the linear component constituting the stent is preferably about 0.04 mm to 0.15 mm, and particularly preferably 0.05 mm to 0.13 mm.

The stent may releasably contain a physiologically active substance. Methods for releasably containing a physiologically active substance in the stent include, for example, a method for coating the surface of the stent with a polymer (for example, a biodegradable polymer) containing a physiologically active substance.

The biodegradable polymer is not particularly limited as long as it is enzymatically or non-enzymatically decomposed in a living body and the decomposed product is nontoxic. Examples of the biodegradable polymer that can be used here include polylactic acid, polyglycolic acid, polylactic acid-polyglycolic acid copolymer, polycaprolactone, polylactic acid-polycaprolactone copolymer, polyorthoesters, polyphosphazene, polyphosphoric acid ester, polyhydroxybutyric acid, polymaleic acid, poly-a-amino acid, collagen, gelatin, laminin, heparan sulfate, fibronectin, vitronectin, chondroitin sulfate, hyaluronic acid, polypeptide, chitin, and chitosan.

In addition, examples of the physiologically active substance to be used here include substances capable of accelerating the liquefaction or metabolism of thrombus or thrombotic complex matter or substances capable of suppressing an increase of thrombus or thrombotic complex matter, substances for suppressing intimal hypertrophy, anticancer agents, immunosuppressing agents, antibiotics, antirheumatics, antithrombotic agents, HMG-CoA reductase inhibitors, ACE inhibitors, calcium antagonists, antilipemia agents, anti-inflammatory agents, integrin inhibitors, antiallergic agents, antioxidants, GP Ilb/Illa antagonists, retinoids, flavonoids, carotenoids, lipid improvers, DNA synthesis inhibitors, tyrosine kinase inhibitors, antiplatelet agents, vascular smooth muscle growth inhibitors, bio-derived materials, interferons, and epithelial cells produced by genetic engineering. A mixture of two or more kinds of the above-mentioned substances and the like may be used.

The substances capable of accelerating the liquefaction or metabolism of thrombus or thrombotic complex matter or substances capable of suppressing an increase of thrombus or thrombotic complex matter are as follows. Examples of the substances capable of accelerating liquefaction of thrombus or thrombotic complex matter include streptokinase, plasminogen activator, urokinase, staphynokinase, lumbrokinase, nattokinase, and their analogs. Examples of the substances capable of suppressing an increase of thrombus or thrombotic complex matter include antiplatelet agents represented by acetylsalicylic acid, ticlopidine, dipyridamole, cilostazol, beraprost sodium, limaprost alfadex, ethyl icosapentate, sarpogrelate hydrochloride, trapidil, clopidogrel, prasugrel, and their analogs, or anticoagulants represented by GP IIb/IIIa antagonist, heparin, and warfarin potassium.

Next, a stent delivery system according to an embodiment of the disclosure here will be described by way of an embodiment shown in the drawings.

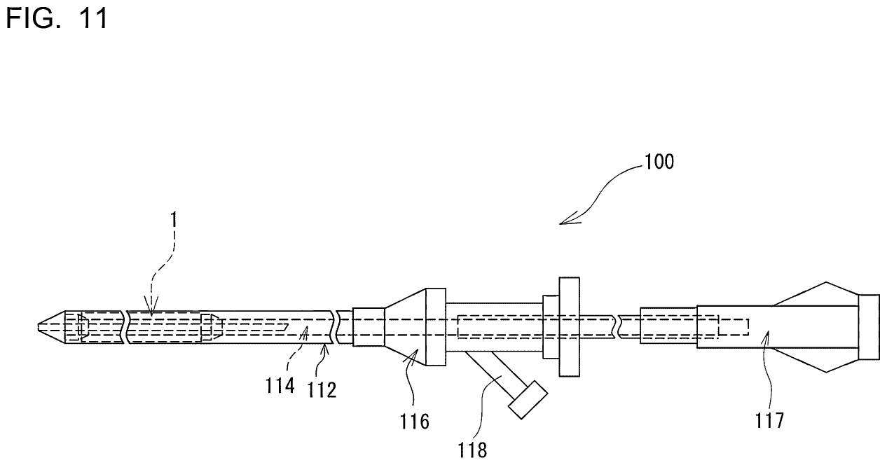

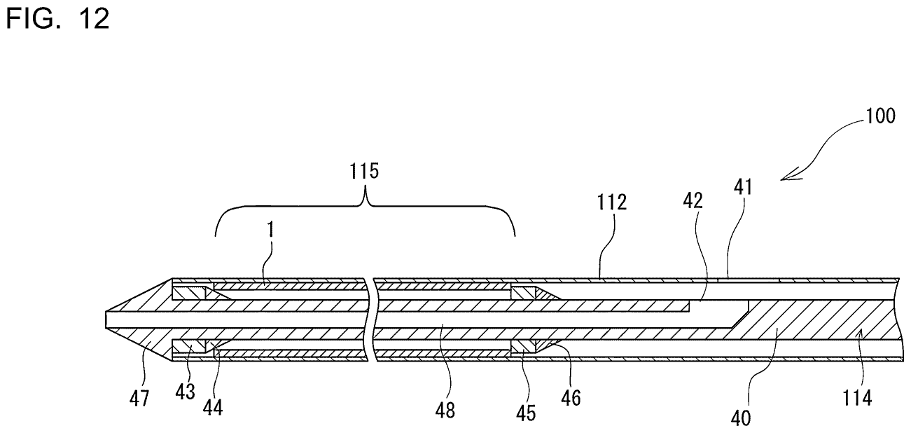

FIG. 11 is a partially omitted front view of the stent delivery system according to the present disclosure. FIG. 12 is an enlarged longitudinal cross-sectional view of the vicinity of a distal portion of the stent delivery system illustrated in FIG. 11.

A stent delivery system 100 according to the present embodiment includes a sheath 112, a stent 1 accommodated in a distal portion of the sheath 112, and an inner tube 114 slidably inserted through or positionable in the sheath 112 for releasing the stent 1 from the distal end of the sheath 112.

The stent delivery system 100 according to the present embodiment employs, as the stent 1, the above-mentioned self-expandable stent that has a substantially cylindrical shape, is compressed in the central axis direction when inserted in a living body, and expands outward to be restorable to the shape before compression when placed in the living body.

The stent delivery system 100 according to the present embodiment includes the sheath 112, the self-expandable stent 1, and the inner tube 114 as illustrated in FIG. 11.

The sheath 112 is a tubular body, and has a distal end and a proximal end that are open as illustrated in FIGS. 11 and 12. The distal opening functions as a release port of the stent 1 when the stent 1 is placed in a stenotic site in the body cavity. The stent 1 is released from the distal opening by sliding movement of the sheath 112 to the proximal side, expands due to the removal of stress loading, and is restored to the shape before compression. The distal portion of the sheath 112 serves as a stent accommodating portion 115 that accommodates the stent 1 therein. In addition, the sheath 112 has a side hole 41 provided on the proximal side with respect to the accommodating portion 115. The side hole 41 is provided to lead out a guide wire therethrough.

The outer diameter of the sheath 112 is preferably about 0.4 mm to 4.0 mm, and particularly preferably 0.5 mm to 3.0 mm. The inner diameter of the sheath 112 is preferably about 0.3 mm to 2.0 mm. The length of the sheath 112 is preferably about 300 mm to 2500 mm, particularly about 300 mm to 2000 mm.

A sheath hub 116 is fixed to the proximal portion of the sheath 112 as illustrated in FIG. 11. The sheath hub 116 includes a sheath hub body and a valve body (not illustrated) that is accommodated in the sheath hub body and holds the inner tube 114 in a slidable and liquid-tight manner. In addition, the sheath hub 116 includes a side port 118 branching obliquely rearward from the vicinity of the center of the sheath hub body. The sheath hub 116 preferably includes an inner tube lock mechanism that restricts the movement of the inner tube 114.

As illustrated in FIGS. 11 and 12, the inner tube 114 includes a shaft-shaped inner tube main body 40, a distal portion 47 provided at the distal end of the inner tube main body 40 and protruding from the distal end of the sheath 112, and an inner tube hub 117 fixed to a proximal portion of the inner tube main body 40.

It is preferable that the distal portion 47 protrudes from the distal end of the sheath 112 and is formed in a tapered shape gradually reducing in diameter toward the distal end as illustrated in FIG. 12. This configuration facilitates insertion into a stenotic site. In addition, it is preferable that the inner tube 114 is provided with a stopper that is provided on the distal side with respect to the stent 1 and restricts the movement of the sheath in the distal direction. The proximal end of the distal portion 47 is contactable to the distal end of the sheath 112, and functions as the above-described stopper.

In addition, the inner tube 114 includes two protruding portions 43 and 45 for holding the self-expandable stent 1 as illustrated in FIG. 12. The protruding portions 43 and 45 are preferably annular. The protruding portion 43 for holding the stent is provided on the proximal side of the distal portion 47 of the inner tube 114. The protruding portion 45 for pressing the stent is provided on the proximal side with respect to the protruding portion 43 for holding the stent by a predetermined distance. The stent 1 is disposed between the two protruding portions 43 and 45. The protruding portions 43 and 45 have outer diameters large enough to contact the stent 1 in a compressed state as described later.

Therefore, the movement of the stent 1 to the distal side is restricted by the protruding portion 43, and the movement to the proximal side is restricted by the protruding portion 45. Furthermore, when the inner tube 114 moves to the distal side, the stent 1 is pushed to the distal side by the protruding portion 45 and ejected from the sheath 112. Furthermore, the proximal side of the protruding portion 45 for pushing the stent is preferably a tapered portion 46 that gradually decreases in diameter toward the proximal side as illustrated in FIG. 12. Similarly, the proximal side of the protruding portion 43 for holding the stent is preferably a tapered portion 44 that gradually decreases in diameter toward the proximal side as illustrated in FIG. 12.

This configuration can prevent the protruding portions from being caught by the distal end of the sheath when the inner tube 114 is projected from the distal end of the sheath 112 to release the stent 1 from the sheath, and then, is accommodated again in the sheath 112. The protruding portions 43 and 45 may be formed of a different member made of an X-ray contrast material. With this configuration, the position of the stent can be accurately recognized under X-ray imaging, which facilitates the procedure.

As illustrated in FIG. 12, the inner tube 114 has a lumen 48 extending from the distal end to at least the proximal side of the stent accommodating portion 115 of the sheath 112 and an inner tube side hole 42 communicating with the lumen 48 on the proximal side with respect to the stent accommodating portion. In the stent delivery system 100 according to the present embodiment, the lumen 48 terminates at the portion where the side hole 42 is formed. The lumen 48 is for permitting a process of inserting one end of a guide wire into the inner tube through the distal end of the stent delivery system 100, partly passing the guide wire through the inside of the inner tube, and extracting the guide wire to the outside from a side surface of the inner tube. The inner tube side hole 42 is located slightly on the distal side of the stent delivery system 100 with respect to the sheath side hole 41. The center of the inner tube side hole 42 is preferably located on the distal side with respect to the center of the sheath side hole 41 by 0.5 mm to 10 mm.

The stent delivery system is not limited to the above-mentioned type, and the above-mentioned lumen 48 may extend up to the proximal end of the inner tube. In this case, the sheath side hole 41 is unnecessary.

The inner tube 114 penetrates the sheath 112 to protrude from the proximal opening of the sheath 112. The inner tube hub 117 is fixed to the proximal portion of the inner tube 114 as illustrated in FIG. 11.

In addition, the stents according to all of the above-described embodiments may include contrast markers at one end and the other end. The contrast marker may be of any type such as an X-ray contrast marker or an ultrasound contrast marker. The marker includes a contrast substance such as an X-ray contrast substance or an ultrasound contrast substance. Preferable examples of a material for forming the marker include gold, platinum, tungsten, iridium, palladium, an alloy thereof, a gold-palladium alloy, platinum-iridium, NiTiPd, and NiTiAu.

The in-vivo indwelling stent disclosed here by way of example includes a plurality of annular bodies each of which is formed into an annular shape using a linear body, and connection portions that connect the annular bodies that are adjacent to each other, wherein a plurality of the annular bodies located on at least a central part of the stent includes a first pattern annular body and a second pattern annular body that are alternately arranged in an axial direction of the stent, the first pattern annular body has an endless zigzag linear shape including a plurality of one-end-side apexes, a plurality of other-end-side apexes, and a plurality of curved linear bodies that connect the plurality of one-end-side apexes and the plurality of other-end-side apexes and that protrude in a first circumferential direction, the second pattern annular body has an endless zigzag linear shape including a plurality of one-end-side apexes, a plurality of other-end-side apexes, and a plurality of curved linear bodies that connect the plurality of one-end-side apexes and the plurality of other-end-side apexes and that protrude in a second circumferential direction opposite to the first circumferential direction, and 80% or more of the one-end-side apexes and the other-end-side apexes of the annular bodies that are adjacent to each other are connected by the connection portions.

Therefore, the stent has excellent retractability by radial compression, blood vessel trackability after being placed in a blood vessel, and satisfactory vasodilating effect.

In addition, the in-vivo indwelling stent according to an aspect of the present disclosure is an in-vivo indwelling stent formed in a substantially tubular shape and including: a plurality of wavy linear bodies extending in an axial direction from one end side to another end side and arranged in a circumferential direction of the stent; and a plurality of connection portions connecting the wavy linear bodies adjacent to each other, the plurality of connection portions extending in a predetermined long axis direction, wherein the wavy linear bodies include a plurality of first linear curved portions whose central parts protrude in a first circumferential direction of the stent and a plurality of second linear curved portions whose central parts protrude in a second circumferential direction opposite to the first circumferential direction, the first linear curved portions and the second linear curved portions being alternately arranged in the axial direction of the stent, the connection portions include a first pattern curved connection portion located between the first linear curved portions of the wavy linear bodies that are adjacent to each other in the circumferential direction, the first pattern curved connection portion connecting a portion on the one end side with respect to the central part of the first linear curved portion that is one of the first linear curved portions and that is located on the first circumferential direction side, and a portion near the central part and on the other end side of the other first linear curved portion, and a second pattern curved connection portion located between the second linear curved portions of the wavy linear bodies that are adjacent to each other in the circumferential direction, the second pattern curved connection portion connecting a portion on the one end side with respect to the central part of the second linear curved portion that is one of the second linear curved portions and that is located on the second circumferential direction side, and a portion near the central part and on the other end side of the other second linear curved portion, a plurality of the first pattern curved connection portions is arranged in the circumferential direction of the stent with central parts protruding in the first circumferential direction, and a plurality of the second pattern curved connection portions is arranged in the circumferential direction of the stent with central parts protruding in the second circumferential direction.

Therefore, the stent has excellent retractability by radial compression, blood vessel trackability after being placed in a blood vessel, and satisfactory vasodilating effect.

In addition, the in-vivo indwelling stent according to an aspect of the disclosure here is an in-vivo indwelling stent formed in a substantially tubular shape and including: a plurality of first pattern annular portions in which a plurality of S-shaped curved linear bodies extending in an axial direction of the stent is arranged in a circumferential direction; and a plurality of second pattern annular portions in which a plurality of inverted S-shaped curved linear bodies extending in the axial direction of the stent is arranged in the circumferential direction, first pattern annular portions and the second pattern annular portions being alternately arranged in the axial direction of the stent, wherein a first pattern annular portion and a second pattern annular portion adjacent to each other in the axial direction among the first pattern annular portions and the second pattern annular portions include an overlapping portion in which an end of the S-shaped curved linear body and an end of the inverted S-shaped curved linear body overlap each other in the circumferential direction between ends of the first pattern annular portion and the second pattern annular portion, the first pattern annular portion and the second pattern annular portion adjacent to each other in the axial direction being connected to each other in the overlapping portion, and in the overlapping portion, one end of the inverted S-shaped curved linear body is connected to a portion located on one end side with a predetermined length from the other end of the S-shaped curved linear body adjacent to the inverted S-shaped curved linear body on one end side in the axial direction of the stent, the other end of the inverted S-shaped curved linear body is connected to a portion located on the other end side with a predetermined length from one end of the S-shaped curved linear body adjacent to the inverted S-shaped curved linear body on the other end side in the axial direction of the stent, one end of the S-shaped curved linear body is connected to a portion located on one end side with a predetermined length from the other end of the inverted S-shaped curved linear body adjacent to the S-shaped curved linear body on one end side in the axial direction of the stent, and the other end of the S-shaped curved linear body is connected to a portion located on the other end side with a predetermined length from one end of the inverted S-shaped curved linear body adjacent to the S-shaped curved linear body on the other end side in the axial direction of the stent.

Therefore, the stent has excellent retractability by radial compression, blood vessel trackability after being placed in a blood vessel, and satisfactory vasodilating effect.

The in-vivo indwelling stent according to the disclosure here is configured as follows.

(1) An in-vivo indwelling stent comprising a plurality of annular bodies each of which is formed into an annular shape using a linear body, and connection portions that connect the annular bodies that are adjacent to each other, wherein
a plurality of the annular bodies located on at least a central part of the stent includes a first pattern annular body and a second pattern annular body that are alternately arranged in an axial direction of the stent,
the first pattern annular body has an endless zigzag linear shape including a plurality of one-end-side apexes, a plurality of other-end-side apexes, and a plurality of curved linear bodies that connect the plurality of one-end-side apexes and the plurality of other-end-side apexes and that protrude in a first circumferential direction,
the second pattern annular body has an endless zigzag linear shape including a plurality of one-end-side apexes, a plurality of other-end-side apexes, and a plurality of curved linear bodies that connect the plurality of one-end-side apexes and the plurality of other-end-side apexes and that protrude in a second circumferential direction opposite to the first circumferential direction, and
80% or more of the one-end-side apexes and the other-end-side apexes of the annular bodies that are adjacent to each other are connected by the connection portions.

This in-vivo indwelling stent includes a plurality of annular bodies each of which is formed into an annular shape using a linear body, and connection portions that connect the annular bodies that are adjacent to each other, wherein a plurality of the annular bodies located on at least a central part of the stent includes a first pattern annular body and a second pattern annular body which are alternately arranged in an axial direction of the stent, the first pattern annular body has an endless zigzag linear shape including a plurality of one-end-side apexes, a plurality of other-end-side apexes, and a plurality of curved linear bodies that connect the plurality of one-end-side apexes and the plurality of other-end-side apexes and that protrude in a first circumferential direction, the second pattern annular body has an endless zigzag linear shape including a plurality of one-end-side apexes, a plurality of other-end-side apexes, and a plurality of curved linear bodies that connect the plurality of one-end-side apexes and the plurality of other-end-side apexes and that protrude in a second circumferential direction, and 80% or more of the one-end-side apexes and the other-end-side apexes of the annular bodies that are adjacent to each other are connected by the connection portions.

Therefore, the stent has excellent retractability by radial compression, blood vessel trackability after being placed in a blood vessel, and satisfactory vasodilating effect.

In addition, the above embodiment may be configured as follows.

(2) The in-vivo indwelling stent described in (1), wherein the connection portions adjacent to each other in a circumferential direction of the stent are inclined in the same direction, and the connection portions adjacent to each other in the axial direction of the stent are inclined in different directions.

(3) The in-vivo indwelling stent described in (1) or (2), further comprising an other-end annular body located at another end, the stent having no other-end-side apex that is a free end except for the other-end annular body.

(4) The in-vivo indwelling stent described in any one of (1) to (3), wherein all of apexes of the annular bodies are connected to the connection portions or adjacent annular bodies except for the apexes located at both ends of the stent.

(5) The in-vivo indwelling stent described in any one of (1) to (4), comprising at least two first pattern annular bodies each of which is the first pattern annular body, wherein a number of the second pattern annular bodies is N−1 or more and N+1 or less when a number of the first pattern annular bodies is N.

(6) The in-vivo indwelling stent described in any one of (1) to (5), wherein
the connection portions include a first pattern connection portion and a second pattern connection portion,
the first pattern annular body includes two curved linear bodies extending from the one-end-side apex to two different other-end-side apexes,
the second pattern annular body includes two curved linear bodies extending from the one-end-side apex to two different other-end-side apexes,
the first pattern connection portion extends to another end side and in the second circumferential direction, and connects the other-end-side apexes of the first pattern annular body and the one-end-side apexes of the second pattern annular body, and
the second pattern connection portion extends to the other end side and in the first circumferential direction, and connects the other-end-side apexes of the second pattern annular body and the one-end-side apexes of the first pattern annular body.

(7) The in-vivo indwelling stent described in any one of (1) to (6), wherein each of the curved linear bodies of the first pattern annular body and each of the curved linear bodies of the second pattern annular body have a substantially arc shape.

(8) The in-vivo indwelling stent described in any one of (1) to (7), wherein central parts of the connection portions are curved and protrude outward of the stent when the stent is expanded.

(9) The in-vivo indwelling stent described in any one of (1) to (8), wherein apexes of the annular bodies and the connection portions are connected with continuity.

The in-vivo indwelling stent is also configured as follows.

(10) An in-vivo indwelling stent formed in a substantially tubular shape, the stent comprising:
a plurality of wavy linear bodies extending in an axial direction from one end side to another end side and arranged in a circumferential direction of the stent; and
a plurality of connection portions connecting the wavy linear bodies adjacent to each other, the plurality of connection portions extending in a predetermined long axis direction, wherein
the wavy linear bodies include a plurality of first linear curved portions whose central parts protrude in a first circumferential direction of the stent and a plurality of second linear curved portions whose central parts protrude in a second circumferential direction opposite to the first circumferential direction, the first linear curved portions and the second linear curved portions being alternately arranged in the axial direction of the stent,
the connection portions include a first pattern curved connection portion located between the first linear curved portions of the wavy linear bodies that are adjacent to each other in the circumferential direction, the first pattern curved connection portion connecting a portion on the one end side with respect to the central part of the first linear curved portion that is one of the first linear curved portions and that is located on the first circumferential direction side, and a portion near the central part and on the other end side of the other first linear curved portion, and a second pattern curved connection portion located between the second linear curved portions of the wavy linear bodies that are adjacent to each other in the circumferential direction, the second pattern curved connection portion connecting a portion on the one end side with respect to the central part of the second linear curved portion that is one of the second linear curved portions and that is located on the second circumferential direction side, and a portion near the central part and on the other end side of the other second linear curved portion,
a plurality of the first pattern curved connection portions is arranged in the circumferential direction of the stent with central parts protruding in the first circumferential direction, and a plurality of the second pattern curved connection portions is arranged in the circumferential direction of the stent with central parts protruding in the second circumferential direction.

This in-vivo indwelling stent is formed in a substantially tubular shape and includes: a plurality of wavy linear bodies extending in an axial direction from one end side to another end side and arranged in a circumferential direction of the stent; and a plurality of connection portions connecting the wavy linear bodies adjacent to each other, the plurality of connection portions extending in a predetermined long axis direction, wherein the wavy linear bodies include a plurality of first linear curved portions whose central parts protrude in a first circumferential direction of the stent and a plurality of second linear curved portions whose central parts protrude in a second circumferential direction opposite to the first circumferential direction, the first linear curved portions and the second linear curved portions being alternately arranged in the axial direction of the stent, the connection portions include a first pattern curved connection portion located between the first linear curved portions of the wavy linear bodies that are adjacent to each other in the circumferential direction, the first pattern curved connection portion connecting a portion on the one end side with respect to the central part of the first linear curved portion that is one of the first linear curved portions and that is located on the first circumferential direction side, and a portion near the central part and on the other end side of the other first linear curved portion, and a second pattern curved connection portion located between the second linear curved portions of the wavy linear bodies that are adjacent to each other in the circumferential direction, the second pattern curved connection portion connecting a portion on the one end side with respect to the central part of the second linear curved portion that is one of the second linear curved portions and that is located on the second circumferential direction side, and a portion near the central part and on the other end side of the other second linear curved portion, a plurality of the first pattern curved connection portions is arranged in the circumferential direction of the stent with central parts protruding in the first circumferential direction, and a plurality of the second pattern curved connection portions is arranged in the circumferential direction of the stent with central parts protruding in the second circumferential direction.

Therefore, the stent has excellent retractability by radial compression, blood vessel trackability after being placed in a blood vessel, and satisfactory vasodilating effect.

In addition, the above embodiment may be configured as follows.

(11) The in-vivo indwelling stent described in (10), wherein the first pattern curved connection portion and the second pattern curved connection portion have a substantially arc shape.

(12) The in-vivo indwelling stent described in (10) or (11), wherein the wavy linear bodies are substantially sinusoidal.

(13) The in-vivo indwelling stent described in any one of (10) to (12), comprising four or more of the wavy linear bodies.

(14) The in-vivo indwelling stent described in any one of (10) to (13), wherein the stent includes the first pattern curved connection portions in all of regions between the first linear curved portions, and includes the second pattern curved connection portions in all of regions between the second linear curved portions.

The in-vivo indwelling stent is also configured as follows.

(15) An in-vivo indwelling stent formed in a substantially tubular shape, the stent comprising:
a plurality of first pattern annular portions in which a plurality of S-shaped curved linear bodies extending in an axial direction of the stent is arranged in a circumferential direction; and
a plurality of second pattern annular portions in which a plurality of inverted S-shaped curved linear bodies extending in the axial direction of the stent is arranged in the circumferential direction, the first pattern annular portions and the second pattern annular portions being alternately arranged in the axial direction of the stent, wherein
a first pattern annular portion and a second pattern annular portion adjacent to each other in the axial direction among the first pattern annular portions and the second pattern annular portions include an overlapping portion in which an end of the S-shaped curved linear body and an end of the inverted S-shaped curved linear body overlap each other in the circumferential direction between ends of the first pattern annular portion and the second pattern annular portion, the first pattern annular portion and the second pattern annular portion adjacent to each other in the axial direction being connected to each other in the overlapping portion, and
in the overlapping portion, one end of the inverted S-shaped curved linear body is connected to a portion located on one end side with a predetermined length from the other end of the S-shaped curved linear body adjacent to the inverted S-shaped curved linear body on one end side in the axial direction of the stent, the other end of the inverted S-shaped curved linear body is connected to a portion located on the other end side with a predetermined length from one end of the S-shaped curved linear body adjacent to the inverted S-shaped curved linear body on the other end side in the axial direction of the stent, one end of the S-shaped curved linear body is connected to a portion located on one end side with a predetermined length from the other end of the inverted S-shaped curved linear body adjacent to the S-shaped curved linear body on one end side in the axial direction of the stent, and the other end of the S-shaped curved linear body is connected to a portion located on the other end side with a predetermined length from one end of the inverted S-shaped curved linear body adjacent to the S-shaped curved linear body on the other end side in the axial direction of the stent.

This in-vivo indwelling stent is formed in a substantially tubular shape and includes: a plurality of first pattern annular portions in which a plurality of S-shaped curved linear bodies extending in an axial direction of the stent is arranged in a circumferential direction; and a plurality of second pattern annular portions in which a plurality of inverted S-shaped curved linear bodies extending in the axial direction of the stent is arranged in the circumferential direction, the first pattern annular portions and the second pattern annular portions being alternately arranged in the axial direction of the stent, wherein a first pattern annular portion and a second pattern annular portion adjacent to each other in the axial direction among the first pattern annular portions and the second pattern annular portions include an overlapping portion in which an end of the S-shaped curved linear body and an end of the inverted S-shaped curved linear body overlap each other in the circumferential direction between ends of the first pattern annular portion and the second pattern annular portion, the first pattern annular portion and the second pattern annular portion adjacent to each other in the axial direction being connected to each other in the overlapping portion, and in the overlapping portion, one end of the inverted S-shaped curved linear body is connected to a portion located on one end side with a predetermined length from the other end of the S-shaped curved linear body adjacent to the inverted S-shaped curved linear body on one end side in the axial direction of the stent, the other end of the inverted S-shaped curved linear body is connected to a portion located on the other end side with a predetermined length from one end of the S-shaped curved linear body adjacent to the inverted S-shaped curved linear body on the other end side in the axial direction of the stent, one end of the S-shaped curved linear body is connected to a portion located on one end side with a predetermined length from the other end of the inverted S-shaped curved linear body adjacent to the S-shaped curved linear body on one end side in the axial direction of the stent, and the other end of the S-shaped curved linear body is connected to a portion located on the other end side with a predetermined length from one end of the inverted S-shaped curved linear body adjacent to the S-shaped curved linear body on the other end side in the axial direction of the stent.

Therefore, the stent has excellent retractability by radial compression, blood vessel trackability after being placed in a blood vessel, and satisfactory vasodilating effect.

In addition, the above embodiment may be configured as follows.

(16) The in-vivo indwelling stent described in (15), wherein, in regions between the S-shaped curved linear bodies and the inverted S-shaped curved linear bodies adjacent to each other in the axial direction, the ends of all the S-shaped curved linear bodies located in a central part of the stent are connected to two of the inverted S-shaped curved linear bodies, and the ends of all the inverted S-shaped curved linear bodies located in the central part of the stent are connected to two of the S-shaped curved linear bodies.

(17) The in-vivo indwelling stent described in (15) or (16), wherein the S-shaped curved linear bodies and the inverted S-shaped curved linear bodies have substantially symmetrical shapes.

(18) The in-vivo indwelling stent described in any one of (15) to (17), wherein a number of the S-shaped curved linear bodies constituting each of the first pattern annular portions and a number of the inverted S-shaped curved linear bodies constituting each of the second pattern annular portions are equal to each other.

(19) The in-vivo indwelling stent described in any one of (15) to (18), comprising at least two first pattern annular portions that are the first pattern annular portions, wherein a number of the second pattern annular portions is N−1 or more and N+1 or less when a number of the first pattern annular portions is N.

(20) The in-vivo indwelling stent described in any one of (1) to (19), the stent being formed into a substantially cylindrical shape, being compressed in a central axis direction when inserted in a living body, and expanding outward to be restored to a shape before compression when placed in the living body.

In addition, the stent delivery system is configured as follows.

(21) A stent delivery system comprising: a sheath; the stent described in (20) accommodated in a distal portion of the sheath; and a shaft that is slidably inserted through the sheath for releasing the stent from a distal end of the sheath.

The detailed description above describes embodiments of a stent and stent delivery system representing examples of the new stent and stent delivery system disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims.

What is claimed is:

1. An in-vivo indwelling stent that extends in an axial direction, has a central portion in the axial direction and is expandable from a contracted state to an expanded state, the in-vivo indwelling stent comprising:
a plurality of annular bodies each of which is constituted by a linear body that is in an annular shape, and connection portions that connect the annular bodies that are adjacent to each other,
a plurality of the annular bodies located on at least the central part of the stent including a first pattern annular body and a second pattern annular body that are alternately arranged in the axial direction of the stent,
the first pattern annular body having an endless zigzag linear shape including a plurality of one-end-side apexes, a plurality of other-end-side apexes, and a plurality of curved linear bodies that connect the plurality of one-end-side apexes and the plurality of other-end-side apexes and that protrude in a first circumferential direction,
the second pattern annular body having an endless zigzag linear shape including a plurality of one-end-side apexes, a plurality of other-end-side apexes, and a plurality of curved linear bodies that connect the plurality of one-end-side apexes of the second pattern annular body and the plurality of other-end-side apexes of the second pattern annular body and that protrude in a second circumferential direction opposite to the first circumferential direction, and
80% or more of the one-end-side apexes and the other-end-side apexes of the first and second pattern annular bodies that are adjacent to each other are connected by the connection portions.

2. The in-vivo indwelling stent according to claim 1, wherein the connection portions adjacent to each other in a circumferential direction of the stent are inclined in a common direction, and the connection portions adjacent to each other in the axial direction of the stent are inclined in different directions.

3. The in-vivo indwelling stent according to claim 1, wherein the stent has one end and an other end at opposite axial ends of the stent, the stent including one end annular body at the one end of the stent and an other-end annular body at the other end of the stent, the stent including plural first pattern annular bodies and plural second pattern annular bodies, all of the one-end-side apexes and the other-end-side apexes of the first and second pattern annular bodies being connected to the connection portions or adjacent annular bodies except for the apexes located at both axial ends of the stent, the stent having no other-end-side apex that is a free end except for the other-end annular body.

4. The in-vivo indwelling stent according to claim 1, wherein the apex of each of the annular bodies has curved outer edges that are gently connected to the end of the connection portion without having a distinct bent portion.

5. The in-vivo indwelling stent according to claim 1, comprising at least two first pattern annular bodies each of which is the first pattern annular body, wherein a number of the second pattern annular bodies is N−1 or more and N+1 or less when a number of the first pattern annular bodies is N.

6. The in-vivo indwelling stent according to claim 1, wherein
the connection portions include a first pattern connection portion and a second pattern connection portion,
the first pattern annular body includes two curved linear bodies of the plurality of curved linear bodies extending from one of the one-end-side apexes to two different other-end-side apexes of the plurality of other-end-side apexes,
the second pattern annular body includes two curved linear bodies of the plurality of curved linear bodies extending from one of the one-end-side apexes to two different other-end-side apexes of the plurality of other-end-side apexes,
the first pattern connection portion extends to another end side and in the second circumferential direction, and connects the other-end-side apexes of the first pattern annular body and the one-end-side apexes of the second pattern annular body, and
the second pattern connection portion extends to the other end side and in the first circumferential direction, and connects the other-end-side apexes of the second pattern annular body and the one-end-side apexes of the first pattern annular body.

7. The in-vivo indwelling stent according to claim 1, wherein each of the curved linear bodies of the first pattern annular body and each of the curved linear bodies of the second pattern annular body have a substantially arc shape.

8. The in-vivo indwelling stent according to claim 1, wherein the connection parts each include a central part, the central parts of the connection portions being curved and protruding outward of the stent when the stent is expanded.

9. The in-vivo indwelling stent according to claim 1, wherein the one-end-side apexes and the other-end-side apexes of the first and second pattern annular bodies and the connection portions are connected with continuity.

10. The in-vivo indwelling stent according to claim 1, the stent being substantially cylindrical in shape, being compressed in a central axis direction when inserted in a living body, and being expandable outward to be restored to a shape before compression.

11. An in-vivo indwelling stent extending in an axial direction and having a substantially tubular shape, the stent comprising:
a plurality of wavy linear bodies extending in the axial direction from one end side to another end side and arranged in a circumferential direction of the stent;
a plurality of connection portions connecting the wavy linear bodies adjacent to each other, the plurality of connection portions extending in a predetermined long axis direction;
the wavy linear bodies including a plurality of first linear curved portions having central parts that protrude in a first circumferential direction of the stent and a plurality of second linear curved portions having central parts that protrude in a second circumferential direction opposite to the first circumferential direction, the first linear curved portions and the second linear curved portions being alternately arranged in the axial direction of the stent;
the connection portions including a first pattern curved connection portion located between the first linear curved portions of the wavy linear bodies that are adjacent to each other in the circumferential direction, the first pattern curved connection portion connecting a portion on the one end side with respect to the central part of the first linear curved portion that is one of the first linear curved portions and that is located on the first circumferential direction side, and a portion near the central part and on the other end side of the other first linear curved portion, and a second pattern curved connection portion located between the second linear curved portions of the wavy linear bodies that are adjacent to each other in the circumferential direction, the second pattern curved connection portion connecting a portion on the one end side with respect to the central part of the second linear curved portion that is one of the second linear curved portions and that is located on the second circumferential direction side, and a portion near the central part and on the other end side of the other second linear curved portion;
a plurality of the first pattern curved connection portions being arranged in the circumferential direction of the stent with central parts of the first pattern curved connection portions protruding in the first circumferential direction, and a plurality of the second pattern curved connection portions being arranged in the circumferential direction of the stent with central parts of the second pattern curved connection portions protruding in the second circumferential direction.

12. The in-vivo indwelling stent according to claim 11, wherein the first pattern curved connection portion and the second pattern curved connection portion have a substantially arc shape.

13. The in-vivo indwelling stent according to claim 11, wherein the wavy linear bodies are substantially sinusoidal.

14. The in-vivo indwelling stent according to claim 11, wherein the stent includes the first pattern curved connection portions in all regions between the first linear curved portions, and includes the second pattern curved connection portions in all regions between the second linear curved portions.

15. An in-vivo indwelling stent extending in an axial direction and having a substantially tubular shape, the stent comprising:
a plurality of first pattern annular portions arranged in a circumferential direction of the stent, each of the first pattern annular portions including a plurality of S-shaped curved linear bodies extending in the axial direction of the stent;
a plurality of second pattern annular portions arranged in the circumferential direction of the stent, each of the second pattern annular portions including a plurality of inverted S-shaped curved linear bodies extending in the axial direction of the stent, the first pattern annular portions and the second pattern annular portions being alternately arranged in the axial direction of the stent;
the first pattern annular portions and the second pattern annular portions that are adjacent to each other in the axial direction including an overlapping portion in which an end of the S-shaped curved linear body and an end of the inverted S-shaped curved linear body overlap each other in the circumferential direction between ends of the first pattern annular portion and the second pattern annular portion, the first pattern annular portion and the second pattern annular portion adjacent to each other in the axial direction being connected to each other in the overlapping portion; and
in the overlapping portion, one end of the inverted S-shaped curved linear body is connected to a portion located on one end side with a predetermined length from the other end of the S-shaped curved linear body adjacent to the inverted S-shaped curved linear body on one end side in the axial direction of the stent, the other end of the inverted S-shaped curved linear body is connected to a portion located on the other end side with a predetermined length from one end of the S-shaped curved linear body adjacent to the inverted S-shaped curved linear body on the other end side in the axial direction of the stent, one end of the S-shaped curved linear body is connected to a portion located on one end side with a predetermined length from the other end of the inverted S-shaped curved linear body adjacent to the S-shaped curved linear body on one end side in the axial direction of the stent, and the other end of the S-shaped curved linear body is connected to a portion located on the other end side with a predetermined length from one end of the inverted S-shaped curved linear body adjacent to the S-shaped curved linear body on the other end side in the axial direction of the stent.

16. The in-vivo indwelling stent according to claim 15, wherein the ends of all the S-shaped curved linear bodies located in a central part of the stent are connected to two of the inverted S-shaped curved linear bodies, and the ends of all the inverted S-shaped curved linear bodies located in the central part of the stent are connected to two of the S-shaped curved linear bodies.

17. The in-vivo indwelling stent according to claim 15, wherein the S-shaped curved linear bodies and the inverted S-shaped curved linear bodies have substantially symmetrical shapes.

18. The in-vivo indwelling stent according to claim 15, wherein a number of the S-shaped curved linear bodies constituting each of the first pattern annular portions and a number of the inverted S-shaped curved linear bodies constituting each of the second pattern annular portions are equal to each other.

19. The in-vivo indwelling stent according to claim 15, wherein a number of the second pattern annular portions is N−1 or more and N+1 or less when a number of the first pattern annular portions is N.

20. A stent delivery system comprising: a sheath; the stent according to claim 19 accommodated in a distal portion of the sheath; and a shaft that is slidably insertable through the sheath for releasing the stent from a distal end of the sheath.
